(19) United States
(12) Patent Application Publication
(10) Pub. No.: US 2004/0229252 A1
(43) Pub. Date: Nov. 18, 2004

(54) Genotyping the T cell receptor

(75) Inventor: Michael A. Siani-Rose, San Francisco, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affmetrix, INC., Santa Clara, CA

(21) Appl. No.: 10/782,339

(22) Filed: Feb. 19, 2004

Related U.S. Application Data
(60) Provisional application No. 60/448,963, filed on Feb. 19, 2003.

(51) Int. Cl. C12Q 1/68

(52) U.S. Cl. 435/6

(57) ABSTRACT

Immune response to pathogens and tumors includes the activation of T cell receptors. Methods for detecting set of polymorphisms in the TCR gene(s) can be very useful for monitoring disease and disease susceptibility. High density nucleic acid arrays may be used to identify single nucleotide polymorphisms in the T cell receptor and the precise T cell receptor species responsible for immunity or self-immune reactions.

### RELATED APPLICTIONS

This application claims priority to U.S. Provisional Application Ser. No. 60/448,963, filed Feb. 19, 2003, the disclosure of which is incorporated herein by reference in its entirety.

### BACKGROUND OF THE INVENTION

The immune system of a mammal is one of the most versatile biological systems as probably greater than 10¹⁰ immunoglobulins and 10¹⁵ T-cell receptors specificities can be produced. Given the T cell receptor's critical role in initiating specific immune responses, it has been suggested that such receptors play a major role in autoimmune disease, cancer, and other T-cell mediated diseases. Much of medical research is directed toward analyzing the immune response repertoire in diseased tissues. Therefore, there is a great need to rapidly detect alterations in the T-cell receptors or immunoglobulins repertoire that may be associated with immunization or with human diseases such as bacterial and viral infections, autoimmune diseases and cancer.

### SUMMARY OF THE INVENTION

In one aspect of the invention, high density oligonucleotide probe arrays are used to detect SNPs or other polymorphism genotypes in the T cell receptor. In preferred embodiments, the method include obtaining a biological sample comprising suitable cells from an individual, extracting nucleic acid from the cells; providing a nucleic acid array comprising probes designed to interrogate at least one pre-determined polymorphism of the T cell receptor; hybridizing the nucleic acids to said array; detecting hybridization complexes; and determining whether polymorphism is present in the T cell receptor gene; and determining the T cell receptor genotype of said individual.

In another aspect of the invention, a method for correlating the presence of at least one selected polymorphism and a susceptibility to a disease is provided. The method includes obtaining a first nucleic acid from a population of individuals with a selected disease and a second nucleic acid from a control population of healthy individuals; providing a nucleic acid array comprising probes designed to interrogate at least one T cell receptor polymorphism; generating a first and second hybridization pattern by hybridizing the first nucleic acid to a first copy of the nucleic acid array and the second nucleic acid to a second copy of the nucleic acid array; and analyzing the first and second hybridization patterns to identify at least one polymorphism that is present in higher frequency in population with individuals with the disease than in population of healthy individuals; and identifying at least one disease-specific polymorphism.

In yet another aspect of the invention, a method of predicting an immune response to a disease, said method comprising establishing a correlation between a T cell receptor genotype and a clinical outcome of the disease; genotyping a patient T cell receptor using a nucleic acid array comprising probes designed to interrogate at least one T cell receptor polymorphism; and determining clinical outcome for said patient based on the patient T cell receptor genotype.

### DETAILED DESCRIPTION

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 6,582,938, 5,242,794, 5,494,810, 4,988,617, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592, 6,632,611 and U.S. patent application Ser. Nos. 09/916,135, 09/920,491 and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574 and 60/403,381.

II. Glossary

An “individual” is not limited to a human being, but may also include other organisms including but not limited to mammals, plants, bacteria or cells derived from any of the above.

Nucleic acids according to the present invention may include any polymer or oligomer of pyrimidine and purine bases, preferably cytosine (C), thymine (T), and uracil (U), and adenine (A) and guanine (G), respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The analogs are those molecules having some structural features in common with a naturally occurring nucleoside or nucleotide such that when incorporated in a nucleic acid or oligonucleotide sequence, they allow hybridization with a naturally occurring nucleic acid sequence The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a single-stranded nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA) in which the constituent bases are joined by peptide bonds rather than phosphodiester linkages. (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide”, “nucleic acid” and “oligonucleotide” are used interchangeably in this application.

The term “fragment,” “segment,” or “DNA segment” refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

Probe: As used herein a “probe” is defined as a nucleic acid capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7 deazaguanosine, inosine, etc.). In addition, a linkage other than a phosphodiester bond may join the bases in probes. Modifications in probes may be used to improve or alter hybridization properties. Thus, probes may be peptide nucleic acids in which the constituent bases are joined by peptide bonds rather than phosphodiester linkages. Other modifications may also be used, for example, methylation or inclusion of a label or dye.

Perfect match: The term “match,” “perfect match,” “perfect match probe” or “perfect match control” refers to a nucleic acid that has a sequence that is designed to be perfectly complementary to a particular target sequence or portion thereof. For example, if the target sequence is 5′-GATTGCATA-3′ the perfect complement is 5′-TATGCAATC-3′. Where the target sequence is longer than the probe the probe is typically perfectly complementary to a portion (subsequence) of the target sequence. For example, if the target sequence is a fragment that is 800 bases, the perfect match probe may be perfectly complementary to a 25 base region of the target. A perfect match (PM) probe can be a “test probe”, a “normalization control” probe, an expression level control probe and the like. A perfect match control or perfect match is, however, distinguished from a “mismatch” or “mismatch probe.”

Mismatch: The term “mismatch,” “mismatch control” or “mismatch probe” refers to a nucleic acid whose sequence is deliberately designed not to be perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at the center of the probe, for example if the probe is 25 bases the mismatch position is position 13, also termed the central position, such that the mismatch is most likely to destabilize the duplex with the target sequence under the test hybridization conditions. A homo-mismatch substitutes an adenine (A) for a thymine (T) and vice versa and a guanine (G) for a cytosine (C) and vice versa. For example, if the target sequence was: 5′-AGGTCCA-3′, a probe designed with a single homo-mismatch at the central, or fourth position, would result in the following sequence: 3′-TCCTGGT-5′, the PM probe would be 3′-TCCAGGT-5′.

Restriction enzymes recognize in general a specific nucleotide sequence of four to eight nucleotides (through this number can vary) and cut a DNA molecule at specific site. For example, the restriction enzyme EcORI recognized the sequence GAATTC and will cut the DNA between the G and the first A. Many different restriction enzymes can be chosen for a desired result. Methods for conducting restriction digests will be known to those skilled in the art. For thorough explanation of the use of restriction enzymes, see for example, section 5, specifically pages 5.2 to 5.32 of Sambrook et al., incorporated by reference above. This method can be used for complexity management of nucleic acid samples such as genomic DNA, see U.S. Pat. No. 6,361,947 which is hereby incorporated by reference in its entirety.

In silico digestion is a computer-aided simulation of enzymatic digests accomplished by searching a sequence for restriction sites. In silico digestion provides for the use of a computer system to model enzymatic reactions in order to determine experimental conditions before conducting any actual experiments. An example of an experiment would be to model digestion of the human genome with specific restriction enzymes to predict the sizes of the resulting restriction fragments.

“Genome” designates or denotes the complete, single-copy set of genetic instructions for an organism as coded into the DNA of the organism. A genome may be multi-chromosomal such that the DNA is cellularly distributed among a plurality of individual chromosomes. For example, in human there are 22 pairs of chromosomes plus a gender associated XX or XY pair.

An “allele” refers to one specific form of a gene within a cell or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

At each autosomal specific chromosomal location or “locus” an individual possesses two alleles, one inherited from the father and one from the mother. An individual is “heterozygous” at a locus if it has two different alleles at that locus. An individual is “homozygous” at a locus if it has two identical alleles at that locus.

“Polymorphism” refers to the occurrence of two or more genetically determined alternative sequences or alleles in a population. A polymorphic marker or site is the locus at which divergence occurs. Preferred markers have at least two alleles, each occurring at frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic or biallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

“Single nucleotide polymorphisms” (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Single nucleotide polymorphisms may be functional or non-functional. Functional polymorphisms affect gene regulation or protein sequence whereas non-functional polymorphisms do not. Depending on the site of the polymorphism and importance of the change, functional polymorphisms can also cause, or contribute to diseases.

SNPs can occur at different locations of the gene and may affect its function For instance: Polymorphisms in promoter and enhancer regions can affect gene function by modulating transcription, particularly if they are situated at recognition sites for DNA binding proteins. Polymorphisms in the 5′ untranslated region of genes can affect the efficiency with which proteins are translated. Polymorphisms in the protein-coding region of genes can alter the amino acid sequence and thereby alter gene function. Polymorphisms in the 3′ untranslated region of gene can affect gene function by altering the secondary structure of RNA and efficiency of translation or by affecting motifs in the RNA that bind proteins which regulate RNA degradation. Polymorphisms within introns can affect gene function by affecting RNA splicing.

The term “genotyping” refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites. For example, at a SNP site, 70 percent of the chromosomes may have a T and the remain 30 percent a C. The two forms T and C are called alleles of the SNP studied and the genotype at this site may be TT, TC or CC.

A “phenotype” refers to any visible, detectable or otherwise measurable property of an organism such as symptoms of, or susceptibility to a disease for example.

An “haplotype” is a combination of multiple alleles or genetic markers at neighboring loci on a single chromosome of a given individual and that do not appear to recombine independently. Estimation of haplotype frequencies from genotype data can be accomplished through statistical algorithms such as the expectation-maximation algorithm or E-M algorithm (Excoffier et al. (1995), Molecular Biology of Evolution, 12:921-927). The E-M algorithm use haplotype frequencies from unambiguous individuals to project and infer haplotypes from the ambiguous individuals.

An “haplotype map” refers to a combination of biallelic markers or biallelic SNPs found in a given individual and which may be associated with a phenotype. For example, an haplotype map can be an individual's genotype for multiple loci or SNPs on a single chromosome.

The term “linkage disequilibrium” refers to a population association among alleles at two or more loci. It is a measure of co-segregation of alleles in a population. Linkage disequilibrium or allelic association is the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

A “population” is a group (usually large group) of individuals.

Human population samples corresponds to samples chosen from a population defined by ethnicity (population of origin) and geography. For example population sample could be chosen from different ethnic group such as: African, African-American, Caucasian, Asian, Asian-American, Chinese, Chinese-American, and also depending on the geography: for example Chinese-American from Hawaii.

An antigen is a compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes.

An autoimmune disease is a disease in which the immune system produces an immune response (e.g. a B cell or a T cell response) against an antigen that is part of the normal host, with consequent injury to tissues. An autoantigen may be derived from a host cell, or may be derived from a commensal organism such as the microorganisms (known as commensal organisms) that normally colonise mucosal surfaces.

III. Genotyping the T Cell Receptor

Effective immune responses against viral pathogens and tumors involve the activation, differentiation and clonal expansion of T cells displaying a variety of effectors and regulatory functions. Recognition of antigens is accomplished through the generation of a large repertoire of different cell surface receptors, called T-cell receptors (TCRs) on T cells. TCRs play key role in various aspects of the immune reaction (to pathogens, vaccines, etc.), including autoimmune diseases, cancer and organ transplantation rejection.

A. Structure of T Cell Receptor

Each receptor is made of up two proteins chains. The most abundant T cells in the blood express a TCR that is a heterodimer of two chains designated as alpha (a) and beta (β). A less abundant T cell receptor consists of a gamma (γ) and delta (δ) chains. The αβ TCRs recognized antigen associated with class I or II molecules of the major histocompatibility complex, whereas the γδ TCRs may recognize free antigen. There are three hypervariable regions in each TCR polypeptide that fold to create the antigen-binding site. The joined V (variable), D (diversity) and J (joining) gene segments encode the third hypervariable site (CDR3). This region shows the highest level of diversity. The TCR β and δ exons are assembled from V, D and J segments while the TCR a and y chains are assembled from V and J segments.

Each V gene is composed of three hypervariable regions (CDR: complementarity determining regions) which are responsible for the antigen binding. CDR1 and CDR2 regions located in the V region interact with the conserved region of the HLA molecule. CDR3 is located at the junction of the V and J domain and interacts with the central region of the bound peptide. Conserved framework regions (FR) flank the CDR regions in the V gene.

There are 57 V gene segments including functional and pseudogenes in the TCRα-TCRδ locus. Forty nine are specific to TCRα (41 functional and 8 pseudogenes), five can be used either for the synthesis of TCRα or TCRδ, and three functional V segments are specific of TCRδ. There are 65 V segments in the TCRβ locus (46 functional, 19 pseudogenes) and 14 in the TCRγ segments (6 functional, 8 pseudogenes).

Analysis of 63 Vβ genes yielded to 279 SNPs in the 55300 bp scanned (i.e. about 1 SNP every 200 bp) (Subrahmanyan et al., Am. J. Hum. Genet., 69:381, 2001). SNPs were distributed throughout the V gene segments. Of the identified SNPs 72 resulted in an amino acod change in the TCRβ locus. The remaining SNPs are believed to have regulatory or structural importance. Similar results were found with the Vα/Vδ locus.

B. T Cell Receptor Polymorphisms and Autoimmune Disease

Autoimmune disorders affect 5% to 7% of the human population and are often characterized by tissue destruction mediated by T cells and causing chronic, incapacitating illness. Although all individuals have immune cells that potentially react with antigens present in their own tissues, these autoreactive cells are normally held back by a complex regulatory mechanism. In individuals who develop autoimmune disease, these regulatory mechanisms are proposed to be somehow defective, which allows autoreactive cells to mount an immunological attack against host tissues. Exemplary autoimmune diseases affecting mammals include rheumatoid arthritis (RA), juvenile oligoarthritis, collagen-induced arthritis, adjuvant-induced arthritis, Sjogren's syndrome, multiple sclerosis (MS), experimental autoimmune encephalomyelitis (EAE), inflammatory bowel disease (e.g. Crohn's disease, ulceritive colitis), autoimmune gastric atrophy, pemphigus vulgaris, psoriasis, vitiligo, type I diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, autoimmune hemolytic anemia pernicious anemia, and the like.

Healthy individuals contain regulatory T cells specific for most expressed T cell receptor variable genes. These regulatory T cells are proposed to normally function to control the activity of T cells that express the corresponding V genes. In healthy individuals, potentially autoreactive T cells are held in check, in part, by these regulatory TCR V-specific T cells. However, in individuals that develop autoimmune disease, there is defective regulatory activity towards T cells that express certain V genes. In the presence of an autoantigen stimulus, this regulatory defect allows oligoclonal expansion of autoreactive T cells that express certain of these V genes, which leads to recruitment of other inflammatory T cells to the involved tissue, leading to tissue damage. In humans, certain VP gene segments have also been suggested to be associated with autoimmune diseases such as rheumatoid arthritis (Paliard X. et al., 1991, Science Vol. 253, pp 325-329; Howell et al., 1991, Proc. Natl. Acad. Sci. USA Vol. 88, pp 10921; Sottini et al., Eur. J. Immunol. 21:461, 1991; Uematsu et al., Proc. Natl. Acad. Sci. USA 88:8534, 1991; Marguerie et al., Immunol. Today 338:336, 1992), Sjogren's syndrome (Sumida et al., J. Clin. Invest. 89:681, 1992), and multiple sclerosis (Ben-Nun et al., Proc. Natl. Acad. Sci. USA 88:2466, 1991; Kotzin et al., Proc. Natl. Acad. Sci. USA 88:9161, 1991; Wucherpfennig et al., Science, 248:1016, 1990; Oksenberg et al., Nature 362:68-70, 1993). Such studies, however, have not been deemed to be conclusive, since these studies have been performed mainly either by the tedious procedure of expanding of antigen-reactive T cell clones and subsequent mRNA analysis, or by PCR of cDNA from diseased tissues. PCR analysis in these studies was limited to only a subset of the Vβ gene segments due to the limited availability of sequences for designing unique primers.

Single nucleotide polymorphisms may be found in both coding and non-coding regions and may be functional or non-functional. Polymorphisms in promoter and enhancer regions can affect gene function by modulating transcription, particularly if they are situated at recognition sites for DNA binding proteins. Polymorphisms in the 5′ untranslated region of genes can affect the efficiency with which proteins are translated. Polymorphisms in the protein-coding region of genes can alter the amino acid sequence and thereby alter gene function. Polymorphisms in the 3′ untranslated region of gene can affect gene function by altering the secondary structure of RNA and efficiency of translation or by affecting motifs in the RNA that bind proteins which regulate RNA degradation. Polymorphisms within introns can affect gene function by affecting RNA splicing. Depending on the site of the SNP and importance of the change, polymorphisms can cause or contribute to diseases.

Hundreds of SNPs have been identified in the TCR loci by Southern blot or direct sequencing of PCR products. Most studies have identified SNPs in the variable gene segments, which are involved in antigenic recognition (Rowen et al., Science 272:1755, 1996; Boysen C. et al., 1996, Immunogenetics, 44: 121), however, only few of these SNPs have been genotyped in the same sample.

To date, disease association studies have been limited, in part, by the restricted number of polymorphisms (e.g., restriction fragment length polymorphisms (RFLP) markers). These studies have generally been uninformative because of both the limited number of defined polymorphisms, and the lack of linkage disequilibrium across the TCR gene region (Robinson and Kindt, Proc. Natl. Acad. Sci. USA 82:3804, 1985). As examples, studies on myasthenia gravis (Smith et al., Ann. N.T Acad. Sci. 505:388, 1987), Graves' disease (Weetman et al., Hum. Immunol. 20:167, 1987), rheumatoid arthritis (Keystone et al., Arthritis Rheum. 31:1555, 1988; Mittenburg et al., Scand. J. Immunol 31:121, 1990), and Type I diabetes (Hibberd et al., Diabetic Med. 9:929, 1992) have suggested a role for TCR polymorphisms. Other studies have failed to find an association (Concannon et al., Am. J. Hum. Genet. 47:45, 1990; Hillert et al., J. Neuroimmunol. 31:141, 1991).

C. Methods

The methods of the presently claimed invention are used to identify and genotype at least 100, 1,000, 5,000, 10,000 SNPs in the TCR gene. In one embodiment an oligonucleotide array is provided with probe sets that are complementary to a plurality of SNPs specific of the TCR genes. The present method usually uses precharacterized polymorphisms. Publicly available databases containing TCR polymorphisms and sequence information may be used to design the probe sets (see for example the website for Single Nucleotide Polymorphism of the National Center for Biotechnology Information). In a preferred embodiment, the probe sets are complementary of the variable region of the TCR genes. Methods for determining the sequence of the variable domain of the TCR are disclosed in U.S. application Ser. No. 10/373,952 which is incorporated herein by reference for all purposes. In a preferred embodiment, allele specific probes and hybridization pattern are used to determine the genotype of the polymorphisms (e.g. haplotype structure) in a target DNA molecule. Allele-specific probes can be designed to hybridize to a segment of target DNA from one individual but do not hybridize to the corresponding segment from another individual due to the presence of different polymorphic forms (alleles) in the respective segments from the two individuals (e.g. see U.S. Pat. No. 6,361,947 incorporated by reference in their entirety for all purposes). Hybridization conditions should be sufficiently stringent such that there is a significant difference in hybridization intensity between alleles, and preferably an essentially binary response, whereby a probe hybridizes to only one of the alleles. For details on the use these arrays for the detection of, for example, SNPs, see U.S. Pat. Nos. 6,368,799, 6,300,063, 5,837,832 and HuSNP Mapping Assay (Affymetrix, Santa Clara, Calif.), all incorporated by reference herein.

In a particular embodiment, probes are designed to distinguish between alleles of a polymorphism. The probes are organized in sets of perfect match and mismatch probes for each allele and for each strand. In a preferred embodiment the mismatch position is the central position which in a 25 mer is the 13^{th} base. In a preferred embodiment the array is designed to comprise probes to at least 1,000, 5,000, 10,000 SNPs that are present in the coding or the non-coding region of the TCR. In a preferred embodiment, SNPs are identified in one or multiple segments of the TCR of an individual or of a population of individuals. In another embodiment, presence of such SNPs in some individuals or some populations is correlated to a reduced effective immune response. In some embodiments analysis of the hybridization is done with a computer system and the computer system provides a determination of which alleles are present.

Identification of SNPs in the TCR genes can be used as markers for the different V segments of the TCR receptor. Additionally, presence of at least one SNP can incapacitate a particular exon and therefore might severely restrict the combinatorics of the TCR potential repertoire. On the other hand, non-synonymous changes in the TCR genes could favor the diversity of the immune repertoire.

Also, groups of adjacents SNPs may exhibit patterns of linkage disequilibrium and haplotypic diversity. Characterization of linkage disequilibrium in TCR genes has been the focus of two groups (Moffatt et al., Hum. Mol. Genet., 9:1011, 2000; Subrahmanyan et al., 2001). Studies showed that significant LD was detectable beyond 100 kbp. Interpopulation differences in SNP frequencies may be used in population-based genetic studies. Haplotype can be consequently identified in different individuals or different populations and compared between populations. Haplotype analysis provide important information for effort to associate TCR polymorphisms in the human population with immune response differences, disease and disease susceptibility.

In some embodiments the present invention provides a pool of unique nucleotide sequences complementary to human TCR SNPs and sequence surrounding SNPs which alone, or in combinations of 2 or more, 10 or more, 100 or more, 1,000 or more, 10,000 or more or 100,000 or more can be used for a variety of applications. In one embodiment probes are present on the array so that each SNP is represented by a collection of probes. The array may comprise between 8 and 80 probes for each SNP. In a preferred embodiment the collection comprises about 40 probes for each SNP, 20 for each allele. The probes may be present in sets of 8 probes that correspond to a PM probe for each of two alleles, a MM probe for each of 2 alleles, and the corresponding probes for the opposite strand. So for each allele there may be a perfect match, a perfect mismatch, an antisense match and an antisense mismatch probe. The polymorphic position may be the central position of the probe region, for example, the probe region may be 25 nucleotides and the polymorphic allele may be in the middle with 12 nucleotides on either side. In other probe sets the polymorphic position may be offset from the center. For example, the polymorphic position may be from 1 to 5 bases from the central position on either the 5′ or 3′ side of the probe. The interrogation position, which is changed in the mismatch probes, may remain at the center position. In one embodiment there are 56 probes for each SNP: the 8 probes corresponding to the polymorphic position at the center or 0 position and 8 probes for the polymorphic position at each of the following positions: −4, −2, −1, +1, +3 and +4 relative to the central or 0 position. In another embodiment 40 probes are used, 8 for the 0 position and 8 for each of 4 additional positions selected from: −4, −2, −1, +1, +3 and +4 relative to the central or 0 position. The probes sets used may vary depending on the SNP, for example, for one SNP the probes may be −4, −2, 0, +1 and +4 and for another SNP they may be −2, −1, 0, +1 and +4. Empirical data may be used to choose which probe sets to use on an array. In another embodiment 24 or 32 probes may be used for one or more SNPs.

In many embodiments pairs are present in perfect match and mismatch pairs, one probe in each pair being a perfect match to the target sequence and the other probe being identical to the perfect match probe except that the central base is a homo-mismatch. Mismatch probes provide a control for non-specific binding or cross-hybridization to a nucleic acid in the sample other than the target to which the probe is directed. Thus, mismatch probes indicate whether hybridization is or is not specific. For example, if the target is present, the perfect match probes should be consistently brighter than the mismatch probes because fluorescence intensity, or brightness, corresponds to binding affinity. (See e.g., U.S. Pat. No. 5,324,633, which is incorporated herein for all purposes.) Finally, the difference in intensity between the perfect match and the mismatch probe (I(PM)-I(MM)) provides a good measure of the concentration of the hybridized material. See PCT No. WO 98/11223, which is incorporated herein by reference for all purposes. In another embodiment, the current invention may be combined with known methods to genotype polymorphism in a wide variety of contexts. For example, the methods may be used to do association studies, identify candidate genes associated with a phenotype, genotype SNPs in clinical populations, or correlate genotype information to clinical phenotypes. One skilled in the art will appreciate that a wide range of applications will be available using 2 or more, 10 or more, 100 or more, 1000 or more, 10,000 or more, 100,000 or more, as probes for polymorphism detection and analysis. The combination of the DNA array technology and the Human TCR SNP specific probes in this disclosure is a powerful tool for genotyping and mapping immune disease loci.

In a preferred embodiment, the polymorphisms and haplotype patterns may be detected in sample DNA from an individual being screened and his DNA may be obtained from any biological sample (other than pure red blood cells). For example, convenient tissue samples include whole blood, semen, saliva, tears, fecal matter, urine, sweat, buccal, skin and hair.

For assays of cDNA and mRNA, the tissue should be obtained from an organ in which the target nucleic acid is expressed. For example, the T cells used can be derived from any convenient T cell source, such as lymphatic tissue, spleen cells, blood, cerebrospinal fluid (CSF) or synovial fluid. A convenient source of T cells to use in the assay are peripheral blood mononuclear cells (PBMC), which can be readily prepared from blood by density gradient separation, by leukapheresis or by other standard procedures known in the art. Tissue could also include brain tissues and neurons wherein TCRβ gene has been shown to be expressed (Syken and Shatz, PNAS, 100:13048, 2003).

A population of cells that contains activated T cells can be obtained from a variety of sources, including the peripheral blood, lymph, and the site of the pathology. The peripheral blood is generally the most convenient source of cells. However, appropriate pathological sites include the CNS (and particularly the cerebrospinal fluid) for multiple sclerosis and other autoimmune neurological disorders; the synovial fluid or synovial membrane for rheumatoid arthritis and other autoimmune arthritic disorders; and skin lesions for psoriasis, pemphigus vulgaris and other autoimmune skin disorders, any of which can be readily obtained from the individual. As available, biopsy samples of other affected tissues can be used as the source of T cells, such as intestinal tissues for autoimmune gastric and bowel disorders, thyroid for autoimmune thyroid diseases, pancreatic tissue for diabetes, and the like.

Depending on the study purpose, it may be desirable to start with a cell population that is partially enriched, or highly enriched, for activated T cells. Methods for enriching for desired T cell types are well known in the art, and include positive selection for the desired cells, negative selection to remove undesired cells, and combinations of both methods.

Enrichment methods are conveniently performed by first contacting the cell population with a binding agent specific for a particular T cell surface activation marker or combination of markers. Appropriate binding agents include polyclonal and monoclonal antibodies, which can be labeled with a detectable moiety. If desired, the T cells can be further contacted with a labeled secondary binding agent specific for the primary binding agent. The bound cells can then be detected, and either collected or discarded, using a method appropriate for the particular binding agent, such as a fluorescence activated cell sorter (FACS), an immunomagnetic cell separator, or an affinity column (e.g. an avidin column or a Protein G column). Other methods of enriching cells by positive and negative selection are well known in the art. DNA, total RNA or mRNA is prepared from the obtained cell population.

Before hybridization to an array in many embodiments the genomic sample is amplified under a given set of amplification conditions. In many embodiments amplification is by PCR using primers flanking a suitable fragment e.g. of 50-500 nucleotides containing the locus of the polymorphisms to be analyzed. The target is usually labeled in the course of the amplification. The amplification product can be RNA or DNA, single straded of double stranded. PCR conditions are standard PCR amplification conditions (see, for example, PCR primer A laboratory Manual, Cold Spring Harbor Lab Press, (1995) eds. C. Dieffenbach and G. Dveksler). Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554,517, and 6,063,603 each of which is incorporated herein by reference in their entireties).

The regions that are identified as being of interest by the genotyping array may then be further analyzed. Resequencing arrays may be designed to identify novel polymorphisms in a sequence of interest and may be designed and synthesized to resequence a particular region. Resequencing arrays are available from Affymetrix, Inc. Santa Clara, Calif., for example, CustomSeqTM arrays may be designed to interrogate regions of 30 Kb or more for sequence variation. Resequencing arrays may be used to discover novel SNPs in a region of interest.

In some embodiments, the disease or disease susceptibility may be selected from the group consisting of Addison's disease, atrophic gastritis, autoimmune hemolytic anemia, autoimmune neutropenia, bullous pemphigoid, Crohn's disease, coeliac disease, demyelinating neuropathies, dematomyositis, Goodpasture's syndrome, Graves' disease, hemolytic anemia, idiopathic thrombocytopenia purpura, inflammatory bowel disease, insulin-dependent diabetes mellitus, juvenile diabetes, multiple sclerosis, myasthenia gravis, myocarditis, myositis, myxedema, pemphigus vulgaris, pernicious anaemia, primary glomerulonephritis, rheumatoid arthritis, scleritis, scleroderma, Sjogren's syndrome, systemic lupus erythematosus, and type I diabetes.

The present invention has utility in identifying polymorphisms, haplotype patterns in biological samples. This information may then be used in any number of ways including, but not limited to, association studies, genetic mapping of phenotypic traits (e.g., disease susceptibility or resistance, drug response, etc.), diagnostics, identification of candidate drug targets, treatment efficacy trials, development of therapeutics, and to reveal the basis for a phenotypic trait.

The polymorphisms and haplotype patterns are useful for the identification of genetic components associated with phenotypic traits (e.g. disease susceptibility or disease resistance). Association studies may be performed for this purpose by determining the genotype of a set of at least one polymorphism for two populations of individuals, one of which exhibits a particular phenotypic trait, and one of which lacks the trait. In another embodiment, the genotypes of more than two populations may be compared, for example by ethnicity. The characteristics of the set of polymorphisms that are compared between the populations include, but are not limited to, the frequency of each genotype of each polymorphism, haplotype patterns that include at least one of the polymorphisms. For example, sets of polymorphisms that occur at a higher or lower frequency in one population than in another indicate areas in the genome where phenotypic trait-related loci may be located. In preferred embodiments, an analysis may be performed by comparing the haplotype structure of a region of interest present in two populations to identify those polymorphisms or haplotype patterns that associate with a phenotypic trait of interest. In some aspects, association between a polymorphism or haplotype pattern and a phenotypic trait can be determined by standard statistical methods.

The above description is illustrative and not restrictive. Many variations of the invention will become apparent to those skill in the art upon review of this disclosure. The scope of the invention should, therefore, be determined not with reference to the above description, but instead be determined with reference with the appended claims along with their full scope of equivalents.

What is claimed is:

1. A method of genotyping the T cell receptor using a high density nucleic acid array comprising:
obtaining a biological sample comprising suitable cells from an individual, extracting nucleic acid from said cells;
providing a nucleic acid array comprising probes designed to interrogate at least one pre-determined polymorphism of the T cell receptor;
hybridizing said nucleic acids to said array;
detecting hybridization complexes; and
determining whether polymorphism is present in the T cell receptor gene; and
determining the T cell receptor genotype of said individual.

2. The method of claim 1 wherein the nucleic acid molecules represent the variable regions of the T cell receptors.

3. A method for correlating the presence of at least one selected polymorphism and a susceptibility to a disease, the method comprising the steps of:
obtaining a first nucleic acid from a population of individuals with a selected disease and a second nucleic acid from a control population of healthy individuals;
providing a nucleic acid array comprising probes designed to interrogate at least one T cell receptor polymorphism;
generating a first and second hybridization pattern by hybridizing the first nucleic acid to a first copy of the nucleic acid array and the second nucleic acid to a second copy of the nucleic acid array; and
analyzing the first and second hybridization patterns to identify at least one polymorphism that is present in higher frequency in population with individuals with said disease than in population of healthy individuals; and identifying at least one disease-specific polymorphism.

4. The method of claim 3 wherein the nucleic acid represent the variable regions of the T cell receptors.

5. A method of predicting an immune response to a disease, said method comprising:
establishing a correlation between a T cell receptor genotype and a clinical outcome of said disease;
genotyping a patient T cell receptor using a nucleic acid array comprising probes designed to interrogate at least one T cell receptor polymorphism; and
determining clinical outcome for said patient based on said patient T cell receptor genotype.

5. The method of claim 4 wherein the disease is an autoimmune disease.
